(19) United States
(12) Patent Application Publication
(10) Pub. No.: US 2016/0103116 A1
(43) Pub. Date: Apr. 14, 2016

(54) Electrophysiological Measuring Arrangement, and Electrophysiological Measuring Method

(71) Applicant: UNIVERSITAET ROSTOCK, Rostock (DE)

(72) Inventors: Oliver Klink, Rostock (DE); Philipp Julian Koester, Rostock (DE); Carsten Tautorat, Rostock (DE); Uwe Scheffler, Broderstorf (DE)

(73) Assignee: UNIVERSITAET ROSTOCK, Rostock (DE)

(21) Appl. No.: 14/786,855

(22) PCT Filed: Mar. 27, 2014
(86) PCT No.: PCT/EP2014/000834

§ 371 Date: Oct. 23, 2015

(30) Foreign Application Priority Data
Apr. 26, 2013 (DE) 10 2013 007 295.6

(51) Int. Cl. G01N 33/487; G01N 27/04

(52) U.S. Cl. CPC G01N 33/48728; G01N 27/04

(57) ABSTRACT

The present invention relates to an electrophysiological measuring arrangement (100) and to an electrophysiological measuring method, in which a carrier (12) made of a carrier material (12′) and an aperture region (10) in the region of the carrier (12) are provided for the controlled sealing accumulation of a biological object (O). The aperture region (10) is formed with at least one aperture (14) and with a wall region (11) which, as the aperture inner wall (11i), surrounds the aperture (14), forming the latter. At least the wall region (11) is formed with a region (20) made of a material (12″) which, at least in terms of the composition thereof, substantially corresponds to the carrier material (12′) outside the aperture region (10) but has an increased concentration of at least one ion species in comparison therewith, with the result that the sealing accumulation of a biological object (O) in the aperture region (10) can be promoted by this increased concentration on or in at least the aperture inner wall (11i) of the aperture (14), wherein the concentration of the at least one material species or ion species is modified to a depth of approximately 10 nm from the top side (12a) or surface of the carrier (12) and, in particular, from the aperture inner wall (11i).

### FIELD OF THE INVENTION

The present invention is concerned with an electrophysiological measuring arrangement as well as with an electrophysiological measuring method, in particular under use of the electrophysiological measuring arrangement according to the invention.

### BACKGROUND OF THE INVENTION

Different methods and arrangements are known that are used in the field of electrophysiology to analyze biological objects—i.e. in particular cells in its broadest meanings, cell organells, oocytes and its fragments—regarding proteins, which are integrated and/or absorbed on respective membranes, and their transport characteristics, wherein also vesicles, liposomes or other more or less artificial systems may be used.

To this end, electrical currents and/or electrical voltages are applied and/or measured between a measuring electrode and a counter electrode, which are arranged between the biological object. The measured currents and/or voltages are intended to give information about the underlying physiological processes, in particular transport processes, conformation changes and the like.

Often, there are comparatively very small signals. To achieve a suitable signal to noise ratio it is therefore necessary to provide high sealing resistances, i.e. as little electrical residual conductivity as possible, across the membrane itself or in the touch or contact region between the membrane of the biological object to be analyzed and the aperture wall.

In the known electrophysiological measuring methods and measuring arrangements it is up to now not possible to control in a sufficient manner the sealing resistance and the electrical residual conductivity between the inside and the outside of the cell—or more generally between an inner and an outer side of the membrane—of the biological object to be measured.

As a consequence, the sealing resistances are generically often to low such that a disadvantageous signal-to-noise ratio is provided.

### SUMMARY OF THE INVENTION

The object of the invention is to provide an electrophysiological measuring arrangement as well as an electrophysiological measuring method, according to which it is possible to promote as reliable as possible accumulation of a biological object to be measured as well as formation and quality of a sealing resistance between the biological object to be measured and the measuring system during the accumulation, in particular to achieve a giga seal, i.e. an accumulation with a sealing resistance in the range of giga ohms.

The object of the present invention is solved by an electrophysiological measuring arrangement with the features of independent claim 1. The object of the present invention is further solved by an electrophysiological measuring method comprising the features of independent claim 10. Further advantageous embodiments are described in the dependent claims.

On the one hand the present invention provides an electrophysiological measuring arrangement comprising a carrier, which is formed at least in its inside with or from a carrier material with a predetermined composition—e.g. predetermined concentrations of material species or ion species—and an aperture region in the region of the carrier—i.e. with a region that comprises or forms at least one aperture or measuring aperture—for controlled sealing accumulation of a biological object, e.g. of a cell, a cell organelle, a vesicle, a liposome, a natural or artificial membrane, e.g. a lipid double layer or the like or a fragment thereof. The aperture region is formed with at least one aperture as well as with a wall region, which forms and surrounds the aperture as aperture inner wall. At least the wall region is formed with a region made of a material, which substantially corresponds at least in its composition to the material of the carrier outside of the aperture region, in particular in the average of the carrier or in the inside of the carrier, but comprises in comparison therewith at least one material species, in particular ion species, with a modified, in particular increased concentration. By this modified, in particular locally increased, concentration of the material species or ion species on or in at least the aperture inner wall of the aperture, the sealing accumulation of a biological object on the aperture region can be promoted. Here, the concentration of the at least one material species or ion species is modified up to a predetermined depth from the top side or surface of the carrier and in particular of the aperture inner wall, in particular up to a depth of about 10 nm.

Hence, it is a core idea of the present invention to modify an in particular to increase in an electrophysiological measuring arrangement with an aperture region, which is formed for sealing accumulation, i.e. for accumulation with high sealing resistance with respect to a biological object to be analyzed, on or in at least a wall region, which forms and surrounds an aperture of an aperture region, the concentration of at least one material species or ion species in comparison with the concentration of the material species or ion species outside of the modified region, i.e. in the underlying material of the carrier outside of the aperture region, in particular in the middle of the carrier and/or in the inside of the carrier.

The change of concentration and in particular the increase of concentration have then, due to a corresponding chemical and/or electrical interaction, the result that also sealing accumulation of a biological object to be analyzed can be controlled and in particular promoted, wherein in particular the probability of accumulation of the biological object to be analyzed on the measuring aperture and/or the sealing resistance between the biological object to be analyzed and the measuring aperture can be increased in comparison with the situation that in an electrophysiological measuring arrangement, which is in other respects identical, the modification of the concentration according to the invention is not present or in comparison with the special case of a completely doped wafer, which has the function of promoting or stabilizing the giga seal, but with deteriorated characteristics of the chip with regard to its suitability for measuring.

Hence, according to the present invention it is possible to support the desired biological object in its accumulation and in the formation of a seal with the aperture and the wall region of the aperture and to improve the strength of the sealing, meaning an increased sealing resistance or a strongly reduced residual conductivity as well as an improved mechanical stability of the seal.

Biological objects which may be used for analyzing may be cells, cell organelles, oocytes, bacteria or a combination or fragments thereof, always in its broadest meaning. Also artificial or partially artificial, substantially biological, structures are conceivable, for example in form of vesicles, liposomes, micelles, membrane fragments or the like, into which proteins have been integrated and/or absorbed in natural or artificial manner.

The objects to be analyzed may be most generically natural or partially or completely artificial biological objects. Moreover, non-biological objects may be analyzed, to analyze e.g. pure lipid structures and modifications thereof. In the following, reference is only made to biological objects, wherein, however, all variants described in this meaning shall be comprised as measuring objects.

In other words, the present invention achieves that selected biological objects to be analyzed are adsorbed with an improved sealing resistance and thereafter measured such that an improved signal-to-noise ratio occurs and such that the accumulation is stabilized mechanically, e.g. with the result of an extended measuring period and an improved reliability with respect to the measuring results.

By choosing the species and/or the height of the concentration, modification type and strength of the interaction, and hence the quality and period of accumulation, may be influenced depending on the charge and/or the structure of the membrane of the biological object on its outer and inner side.

The at least one material species or ion species may be or may have been chosen from the group comprising protons (H⁺), halide ions, in particular fluoride ions (F⁻), double charged ions, in particular double charged metal cations, in particular Be²⁺, Sr²⁺, Ca²⁺ and Mg²⁺. However, the present invention is not limited to these species, generally all material species are conceivable, e.g. to allow for given specific surface situations of a biological object to be measures, e.g. to achieve also in the presence of glycocalyx structures or other situations high sealing resistances and mechanically stable accumulations.

Increasing the concentration of the at least one material species or ion species may be spatially restricted such to a local region of the wall region that the material of the carrier comprises in at least one region outside of the aperture region no increased concentration of the at least one material species or ion species.

The concentration of the at least one material species or ion species may be formed to be increased by means of an implantation, in particular by means of a plasma process, a sputter process, or an ion beam process. Conceivable are, however, also other implantation or doping methods.

According to an embodiment of the measuring arrangement according to the present invention the aperture region is formed in the region of a carrier, which comprises a top side and a bottom side. Here, a respective wall region, which forms an aperture, may protrude partially or completely with respect to the top side and/or with respect to the bottom side of the carrier. The provided carrier may also be denoted as basis, substrate or base substrate. Providing such a substrate or such a carrier stabilizes the measuring arrangement and in particular the arrangement of the arranged biological object to be analyzed mechanically as such and allows a microscopic division of the measuring arrangement with respect to the electrolyte bath underlying the measurement in the meaning of a division of a measuring cuvette or wet cell in compartments with measuring and counter electrodes.

A respective aperture forming wall region may also be formed to be integrated in the inner wall of the hole in combination with the electrode arrangement.

Based on the substrate or the carrier one or several apertures with corresponding wall regions may then be formed in a protruding or sticking out manner with respect to the top side.

Alternatively these may also be formed terminating flush with the top side and directed to the inside such that they protrude from the bottom side of the carrier or the substrate. This is, however, not mandatory and may be omitted for a respective thickness of the membrane.

The dimension of the respective protrusion influences the inner wall of the respective wall region and hence the available interaction area with the membrane of the biological object. Selecting the dimension of the protrusion allows moreover an adaption to the respectively available measuring objects, for example with regard to their form or number, in the measuring solution.

The carrier may also be formed as—in particular planar—plate element with front or top side and with back or bottom side. Other geometries are also conceivable.

Alternatively, deviations from the plate form are also allowed, e.g. by choosing the form of a pipette, e.g. meaning a classical patch pipette.

The wall region, which forms an aperture, may also be formed according to a curved surface area or a combination of curved surface areas. To this end, the surface area of a cylinder, a prism, a truncated cone and/or a truncated pyramid may be used, respectively, with respecting wall thickness.

Regarding the form of the wall region for formation of the aperture various possibilities may be chosen, too. These may be selected depending on the form and further—e.g. mechanical, geometrical and/or electrical—characteristics of the biological objects to be analyzed.

Further, the wall region forming an aperture may be formed from an edge or edge region such that the aperture is quasi formed as a planar hole in the underlying substrate and such that in this process the region of modified concentration with or from the material with modified concentration is embedded in the wall region of the planar hole to promote accumulation and sealing and to promote therewith the sealing resistance.

A wall region forming an aperture may be formed with or from a material of the group of materials that comprise glass, quartz glass, silicon, carbon and combinations and derivatives thereof. Also with respect to the selection of materials the characteristics of the underlying biological objects may be taken into account, for example with respect to the surface structure or surface charge of the outer surface of the membrane and/or the inner surface of the membrane of the biological objects, for example also to support a particularly strong accumulation and hence increasing of the sealing resistance of the seal.

The diameter of the aperture and in particular the inner diameter of an or the aperture forming wall region may have a value in the range of about 0 μm to about 50 μm, in particular in the range from about 1 μm to about 50 μm. An or the aperture forming wall region may also have a height or depth in the range from about 0 μm to about 20 μm above the top side or the bottom side of the carrier or the substrate.

The indicated dimensions with respect to height or depth of the wall region and its diameter may further be oriented on the geometrical features of the biological objects to be analyzed, in particular on their size and their mechanical membrane characteristic, and may consequently have different values as the ones concretely indicated above.

For forming a measuring circuit a measuring electrode may be provided in the region of or within the aperture wall in a region on the back or bottom side of the carrier or substrate.

A counter electrode may be provided outside of the aperture and in the region on the front side or top side of the carrier.

Measuring electrode and counter electrode are preferably arranged on opposite sides of the carrier or substrate or the measuring aperture, however, such that after accumulation of a preferably biological object to be measured the object is arranged between the electrodes and virtually separates these electrically after formation of a suitable seal, preferably with a very high resistance, which is ideally larger than 1 giga ohm.

Thus, the underlying structure of the electrophysiological measuring arrangement according to the present invention comprises according to this embodiment, in particular, providing of a measuring electrode arrangement and a counter electrode arrangement, between which an electrical current and/or an electrical voltage may be measured, wherein between the measuring electrode arrangement and the counter electrode arrangement the biological object to be measured is or has been arranged in the region of the aperture and its wall region such that due to the sealing accumulation the residual conductivity, i.e. the conductivity between the membrane of the biological object and the wall region of the aperture, is as small as possible such that the actually measured electrical currents and/or electrical voltages can be considered to be based on the characteristics of the membrane of the biological object, for example on transport processes, on charge replacements within or across the membrane, on substrate binding or release or the like.

According to a further aspect of the present invention an electrophysiological measuring method is provided. It is carried out, in particular, using an electrophysiological measuring arrangement according to the present invention. For controlling, and in particular for promoting, the sealing accumulation of a biological object to be measured on an aperture of an aperture region at least a region of the inner wall of a wall region forming the aperture is or has been formed in a controlled manner with respect to at least one material species or ion species with an accordingly suitably increased concentration with respect to the concentration of the at least one material species or ion species in the carrier outside of the aperture region, and in particular in the average of the carrier and/or in the inside of the carrier.

In the measuring method according to the present invention due to the modified concentration there may therefore also be a direct or indirect manipulation of molecules—of the electrolyte surroundings and/or of the membrane to be accumulated—in the neighborhood of the wall.

It is, hence, accordingly a core aspect, on which the electrophysiological measuring method according to the present invention is based, to modify and in particular increase in a controlled manner the concentration of at least one material species or ion species at least on or in the aperture forming wall region, wherein due to this via interactions with the biological object and/or the electrolyte surroundings a sealing accumulation is promoted and the sealing resistance is increased.

According to an alternative view of the present invention the focus is the—in particular lateral—localization of the modification of the concentration of the at least one material species or ion species to the region of the aperture.

On the one hand, an electrophysiological measuring arrangement is formed, too, with a carrier of a carrier material and with an aperture region in the region of the carrier—i.e. with a region, which comprises or forms the at least one aperture or measuring aperture—for sealing accumulation of a biological object, e.g. a cell, a cell organelle, a vesicle, a liposome, a natural or artificial membrane, e.g. a lipid double layer or the like or a fragment thereof, in a controlled manner. The aperture region is formed with at least one aperture as well as with a wall region, which forms and surrounds the aperture as aperture inner wall. The wall region is formed with a region made of a material, which corresponds at least in its composition substantially to the carrier material outside of the aperture region, but has in comparison therewith at least one material species, in particular ion species, with a locally modified, in particular locally increased, concentration. Due to this locally modified, in particular locally increased, concentration of the material species or ion species on or in the aperture inner wall of the aperture it is possible to promote sealing accumulation of a biological object on the aperture region.

Hence it is the core idea of this alternative view of the present invention to modify and in particular to increase in an electrophysiological measuring arrangement with an aperture region, which is formed for sealing accumulation, i.e. for accumulation with high sealing resistance, with respect to a biological object to be analyzed, on or in a wall region, which forms and surrounds an aperture of an aperture region, locally the concentration of at least one material species or ion species in comparison with the concentration of the material species or ion species in the underlying carrier material outside of the aperture region.

The local concentration change and in particular the local increase of concentration has then due to an according chemical and/or electrical interaction the result that also the sealing accumulation of a biological object to be analyzed can be controlled, and in particular be promoted, wherein in particular the probability of accumulating the biological object to be analyzed on the measuring aperture and/or the sealing resistance between the biological object to be analyzed and the measuring aperture can be increased in comparison with the situation in which in an otherwise identical electrophysiological measuring arrangement the modification of concentration according to the present invention is not present.

The concentration of the at least one material species or ion species may also according to this alternative view of the present invention be modified up to a predetermined depth, e.g. of about 10 nm from the surface of the aperture inner wall. However, also other locally limited layer depths or—in particular in case of lateral locality—also a continuous implantation of the aperture wall is conceivable.

According to a further aspect of the alternative view of the present invention there is provided on the other hand also an electrophysiological measuring method. It is carried out in particular using an electrophysiological measuring arrangement according to the alternative view of the present invention. For controlling, and in particular promoting, of sealing accumulation of a biological object to be measured on an aperture of an aperture region the inner wall of a wall region forming the aperture is or has been formed with respect to at least one material species or ion species in a controlled manner with an accordingly suitably locally increased concentration with respect to the concentration of the at least one material species or ion species in the carrier outside of the aperture region.

Also according to this measuring method according to the present invention due to the locally modified concentration a direct or also indirect manipulation of molecules—of the electrolyte surroundings and/or the membrane to be accumulated—in the neighborhood of the wall may be provided.

Accordingly it is, hence, a core aspect, on which the electrophysiological measuring method according to the present invention is based, to locally modify and in particular increase in a controlled manner the concentration of at least one material species or ion species on or in the wall region forming the aperture, wherein due to this via interactions with the biological object and/or the electrolyte surroundings a sealing accumulation is promoted and the sealing resistance is increased.

In the measuring methods according to the present invention and with the measuring arrangements according to the present invention measuring signals may be measured, in particular also in capacitive manner, to determine e.g. single channel activities.

These and further aspects of the present invention will be described on basis of the accompanying drawings.

### BRIEF DESCRIPTION OF THE FIGURES

FIGS. 1A, 1E show in sectional side view a first or an alternative second embodiment of the electrophysiological measuring arrangement according to the present invention, in which the wall region of an aperture protrudes above the top side of an underlying carrier.

FIGS. 1B-1D show in schematic and sectional top view different forms of cross-sections of an aperture and the respective underlying wall region.

FIGS. 2A-2D show in schematic and sectional side view details of various embodiments of formation of the modified concentration on or in the wall region of an aperture.

FIGS. 3-7 show in analog manner as FIG. 1A in schematic and sectional side view various embodiments of an electrophysiological measuring arrangement according to the present invention, in which a respective aperture is formed by wall regions, that correspond to curved surface areas of different geometrical bodies.

FIG. 8 shows an electrophysiological measuring arrangement according to an embodiment of the present invention, which is formed in the manner of a so-called patch pipette.

FIGS. 9A-9E show in schematic and sectional side view different aspects of the use of the electrophysiological measuring arrangement according to the present invention.

FIGS. 10-12 show in schematic and sectional side view details of the accumulation of a biological object on respective embodiments of the electrophysiological measuring arrangement.

### DETAILED DESCRIPTION OF THE EMBODIMENTS

In what follows embodiments of the present invention will be described in detail. All embodiments of the invention and also their technical features and characteristics may be composed isolated and optionally on its own and may be combined with each other arbitrarily and without limitation.

In what follows structurally and/or functionally equal, similar or equally acting features or elements will be designated in connection with the figures with the same reference signs—as long as nothing else is indicated.

A detailed description of these features or elements will not be reiterated for every appearance of them.

First reference is taken to the drawings in general terms.

In electrophysiology amongst others the patch clamp technology is used, e.g. for carrying out ion channel analysis for medicament testing. In the manual patch clamp method and its refinements electrical currents and voltages may be measured—e.g. on single cells—which are generated in the membranes of biological cells—e.g. from ion channels.

Due to the increasing importance of electrophysiological analysis and due to the personal and temporal effort for its execution there is a large demand for automated electrophysiological measuring techniques and in particular for planar patch clamp and further automated patch clamp or APC systems.

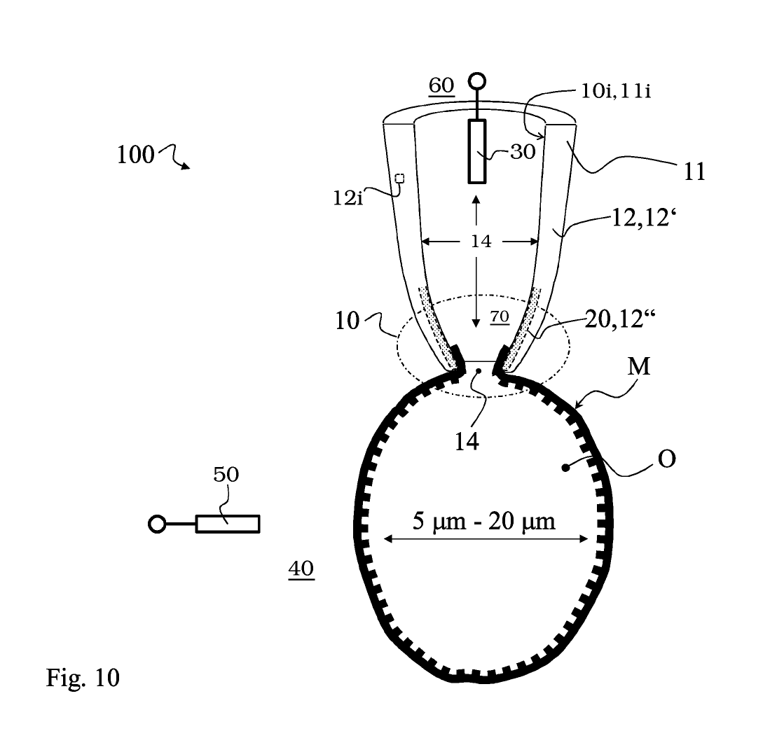

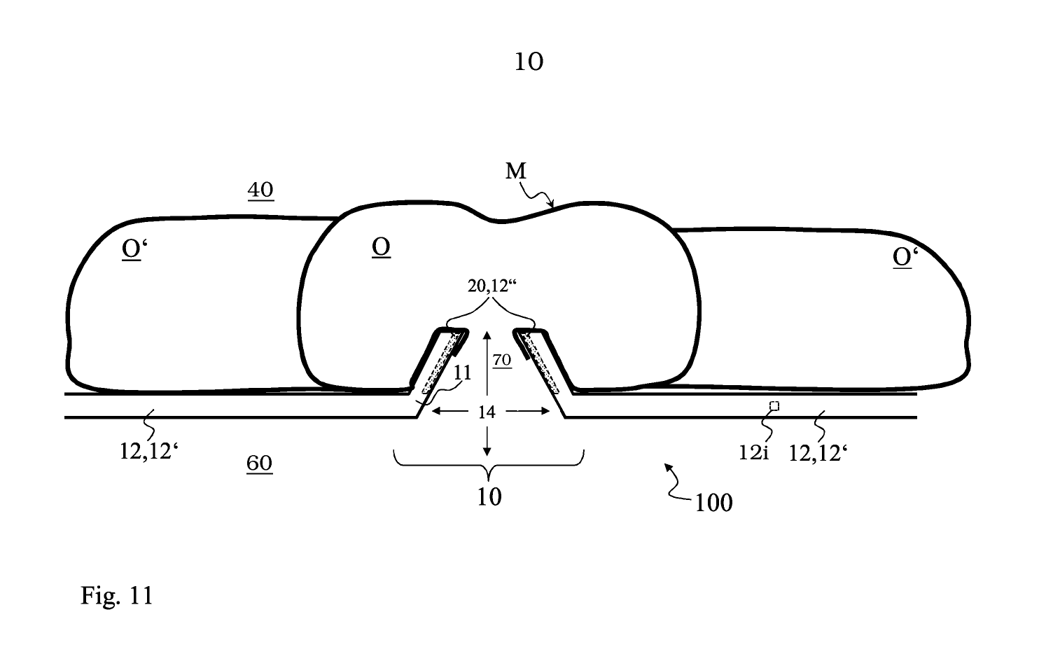

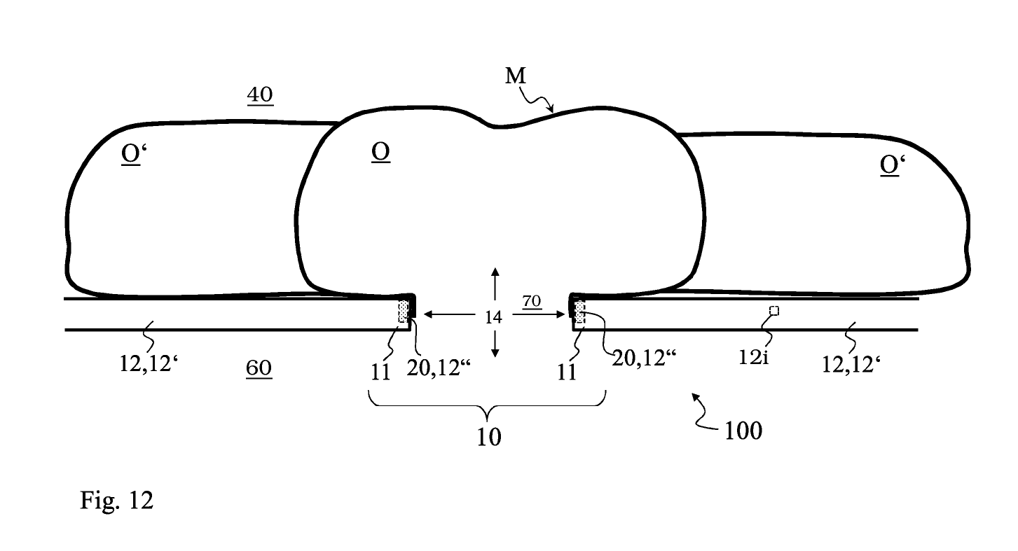

Functioning of a manual patch clamp method and of APC systems is basically the same. In both types of systems the necessity for forming a high ohmic sealing resistance between the measuring object O, e.g. a cell O, and the measuring arrangement 100 is problematic. This is also called the necessity of a so-called giga seal, i.e. of a sealing resistance in the giga-ohm range. To this end, the electrical isolation—e.g. of the inside of the cell to its outside—is e.g. described by a patch pipette, as is shown e.g. in FIG. 10, or for a planar cell arrangement according to an APC scheme according to FIGS. 11 and 12.

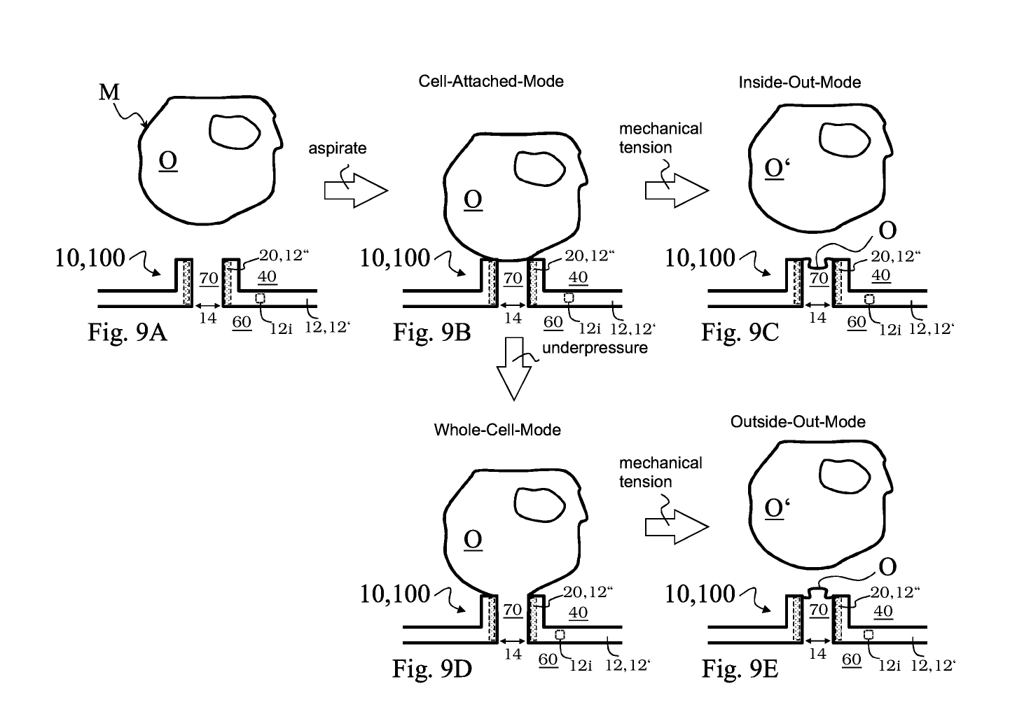

By means of a patch pipette a cell is aspirated as biological object O, if necessary. A small, limited part of the cell membrane M, which is denoted as patch, is sucked in with a small under-pressure. Due to this the measuring surface, in the cell-attached-measurement according to FIG. 9B, or the cell inside, in a whole-cell-measurement according to FIG. 9D, is electrically sealed from the outside of the cell in the mega- to giga-ohm range. The cell attached configuration allows current measurement also in single ion channels of the cell membrane. In manual patch clamp as well as in APC systems the giga seal rate as well as the sealing resistance is a measure for the quality of the possible ion channel measurement. Up to now a 100% giga seal rate has not been achieved.

The formation of the giga seal depends on many parameters, which are not accessible up to now to an active manipulation. Hence a real control of the giga seal during the accumulation and measurement process is lacking.

In the manual patch clamp methods up to now a sufficiently high giga seal rate has only been achieved with freshly manufactured glass pipettes. In the development of chips for APC systems care has to be taken to reduce the surface roughness and to avoid sharp edges. Further, a careful combination of intra and extra cellular buffers may lead to an improvement of the giga seal. However, due to the necessity to use physiological buffers this allows only for limited possibilities of control and manipulation.

The present invention is based on the insight that negative or positive charges on or in the carrier 12 and in particular on or in the inner wall 11i of a measuring aperture 14, e.g. also a patch pipette, can support the formation of a giga seal and can improve the giga seal itself. By a selective and e.g. also space resolved modification, and in particular by increasing, of the concentration of specific material species and in particular of ions the local charge density is—by means of positive or negative charges—on or in the inner wall 11i of the measuring aperture 14 respectively modified, i.e. in particular increased, and the processes for establishing and/or stabilizing a giga seal can be influenced by direct and if necessary local modification on or in the surface, e.g. by a material implantation into the aperture wall, e.g. by means of a plasma.

The control of the charge density on or in the pipette wall or aperture wall 11 is effected according to the present invention by the modification, and in particular by increasing, of the concentration of a material species or ion species at least in a region 20 of the wall region 11 of the aperture region 10 such that at least in the measuring aperture wall 11 and therein locally in the region 20 the material 12″ of the carrier 12 with respect to the at least one material species or ion species a modified, and in particular increased, concentration is formed in comparison with the concentration of the respective species in the material 12′ of the carrier 12 outside of the region 20 and hence outside of the aperture region 10, in particular in comparison with the concentration in the inside of the carrier 12 and/or of an average of the carrier 12. In particular, together with applying a specific voltage on measuring and counter electrodes 30, 50 the described effects and advantages are achieved according to the present invention.

These are in particular and amongst others:
the improvement of the giga seal with respect to the temporal formation and the height of the giga seal, i.e. with respect to the sealing resistance,
the spatially resolved increasing of concentration, in particular of bivalent ions or of protons, and
the use for purposeful adhesion and for growth of cells,
   for each variance of the patch clamp technology and cell culture, respectively.

Together with the development of an APC system for cell networks this invention is particularly advantageous, as due to the use of non-optimal materials formation of a good giga seal is reduced. An improvement of the giga seal or increasing of the giga seal probability provides a principle advantage, if cells overgrow patch pipettes e.g. of APC systems over a longer time period.

The present invention is based amongst others also on the insight that bivalent ions and/or protons have a concentration dependent influence on the temporal formation, the amount and the stability of the giga seal and of the sealing resistance. This is true depending on the situation also for other implanted species which might be used according to the invention, if necessary.

The object of the invention is therefore amongst others the selective and in necessary space resolved or local modification or increasing of the concentration of ions or protons at least in or on the inner wall of the measuring aperture, in order to influence therewith processes for establishing of the giga seal positively. This cannot be ensured by the use of conventional physiological measuring buffers.

The material species and in particular the ions and/or protons may e.g. be implanted by a specific physical plasma process on or inside the surface of the material—i.e. the inner wall of the measuring aperture. Conceivable is also the implantation of a material that then concentrates bivalent ions and/or protons on the surface.

The invention serves therefore amongst others also for the improvement of the material characteristics of the materials required by chip technology—e.g. of glass or silicon or variants thereof—with respect to the formation of a giga seal.

The—in particular local—change of concentration and in particular increase of concentration of e.g. ions or protons, which is allowed by the present invention, is up to now not known and results in an increase of the giga seal probability as well as in the increase of the measured sealing resistance values at the position of the concentration modification, i.e. for example at the location of implantation, as well as in an increased mechanical stability of the seal.

The comparatively high local concentration change and consequently the accompanying local charge density, if necessary, allowed by the present invention, may it be negative or positive, cannot be achieved conventionally.

Accordingly, the present invention relates to an electrophysiological measuring arrangement 100 as well as an electrophysiological measuring method, according to which the sealing accumulation of a biological object O to be analyzed on a carrier 12 of the measuring arrangement 100 can be controlled and in particular promoted, by providing at least on or in a region 20 of a wall region 11, which forms an aperture 14 of an aperture region 10, a material 12″, which substantially corresponds at least in its composition to the material 12′ of the carrier outside of the aperture region 10 and in particular to the material of the carrier 12 in its inside 12i or in its spatial average, but which has in comparison therewith at least one material species or ion species of a—if necessary locally—increased concentration such that by this—if necessary locally—increased concentration on or in the aperture inner wall 11i of the aperture 14 the sealing accumulation of a biological object O on the aperture region 10 can be promoted, in order to, hence, promote via interaction of the membrane of the biological object O to be analyzed the sealing accumulation in a controlled manner. Here, the depth or layer thickness Δ measured from the surface or top side 12a of the carrier 12, up to which the concentration modification of the at least one material species or ion species extends, is preferably in the range of about 10 nm.

Now the drawings will be detailed.

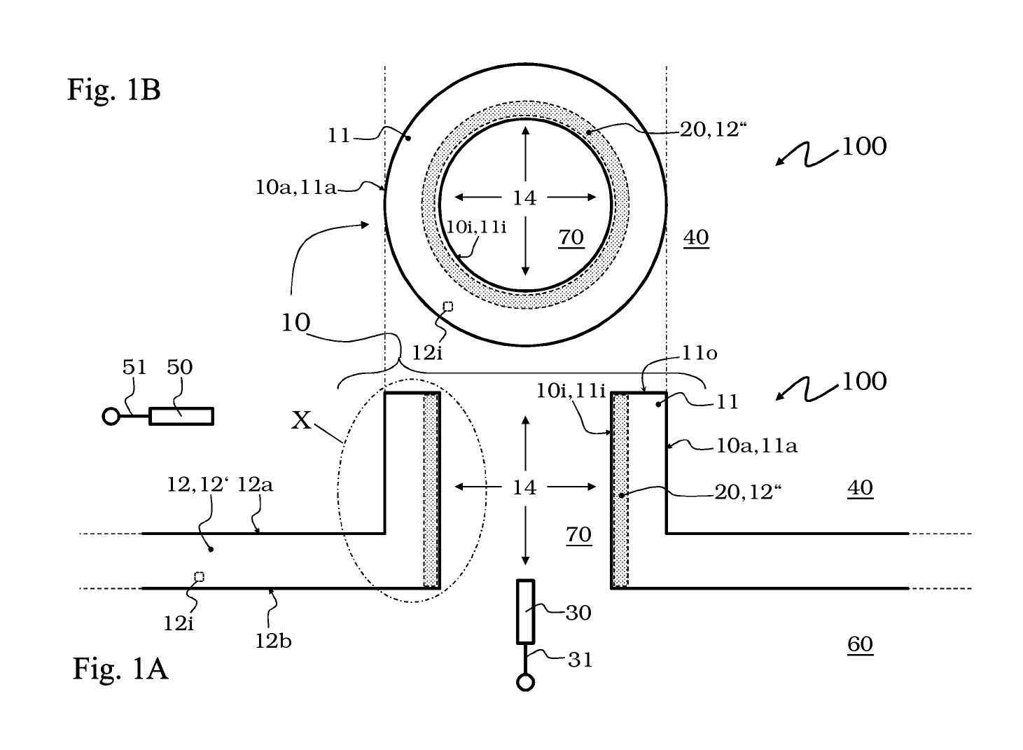

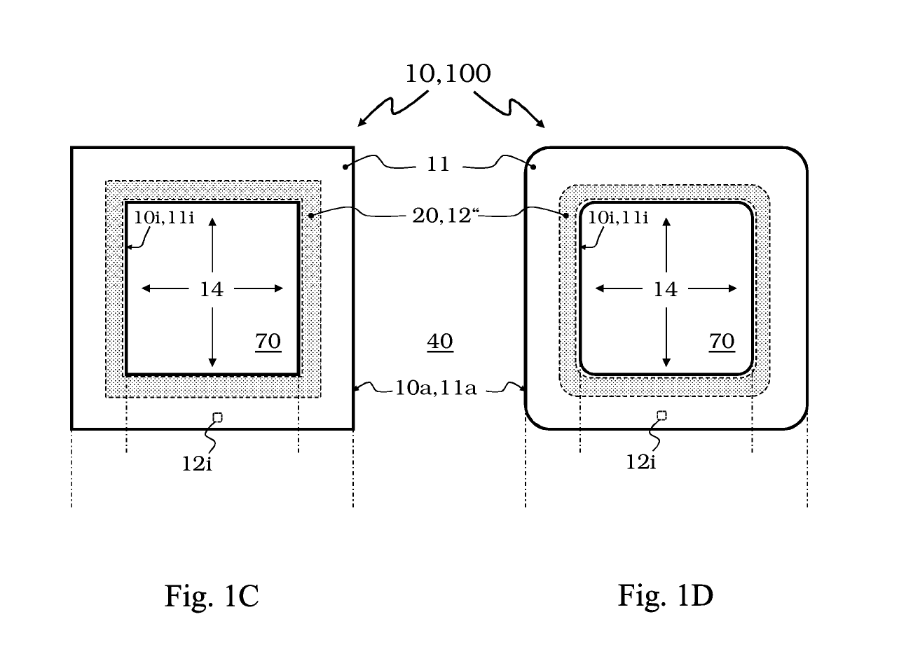

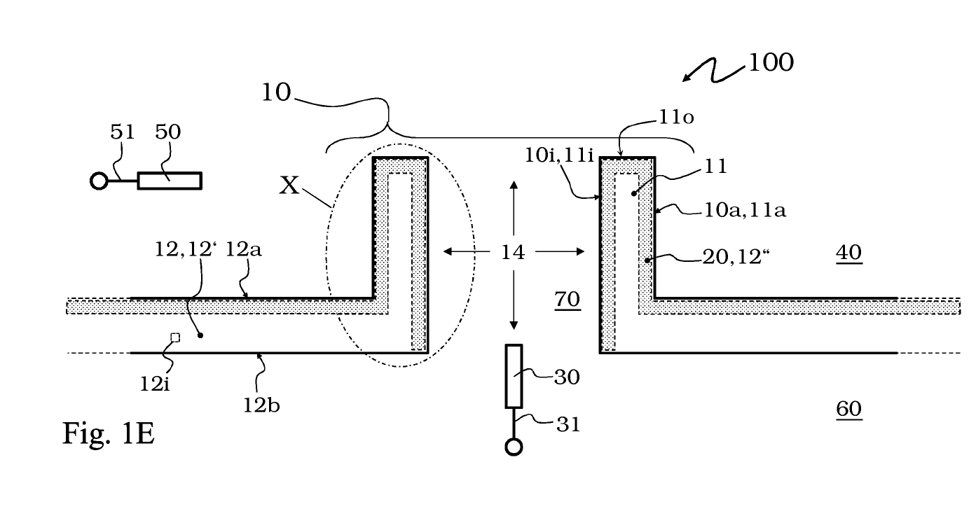

FIG. 1A shows in schematic and sectional side view a first embodiment of the electrophysiological measuring arrangement 100 according to the present invention.

A basic element of this embodiment is the carrier 12, which may also be denoted as base 12 or substrate 12. This carrier 12 divides an electrolyte bath 40, 60, 70, which is provided during the measurement, in at least two compartments, wherein the first compartment 60 faces the bottom side 12b of the carrier 12 or substrate 12 and wherein the second compartment 40 faces the top side 12a of the carrier 12 or substrate 12.

In the carrier 12 a so-called aperture region 10 is embedded. This comprises at least one aperture 14, namely e.g. in the manner of a through hole, which extends locally completely through the carrier 12 in its thickness or layer thickness direction, i.e. in the direction from the top side 12a to the bottom side 12b. In the region of the aperture 14 there is also provided a part 70 of the electrolyte bath 40, 60, 70.

Considered altogether there exists hence between the top side 12a and the bottom side 12b a fluid mechanical connection via the aperture 14 of the aperture region 10 and accordingly also an electrical connection via the conductivity, which is provided if necessary, of the electrolyte bath, which may often be a physiological solution during application. In the embodiment according to FIG. 1A a counter electrode 50 connected via a line 51 is arranged in the upper compartment 40 as counter of a measuring electrode 30, which is provided on the bottom side 12b of the carrier 12. The measuring electrode 30 is therefore located in the bottom side compartment 60 of the electrolyte bath 40, 60, 70 and is connected with a line 31. The lines 51, 31 to the counter electrode 50 and the measuring electrode 30, respectively, are each isolated and lead to a corresponding control and measuring circuit, which is not illustrated.

The substrate 12 or the carrier 12 is formed from a carrier material 12′, which has at least in the inside 12i or in the wall of the carrier 12 a specific composition. At least on or in the wall region 11 of the aperture region 10 a region 20 is provided, which is formed with or from a material 12″, which corresponds substantially to the carrier material 12′ outside of the aperture region 10 and therefore outside of the region 20 and in particular to the material in the inside 12i of the carrier or to the material of the carrier 12 in its spatial average at least in its composition, but has in comparison therewith at least one material species or ion species with a—if necessary locally or laterally locally—modified or increased concentration.

Providing and forming of such a region 20—which will be denoted in what follows also as concentration modified region 20 and which is illustrated in the figures by a dashed frame filled with dots—having in comparison with the remaining carrier material 12′ or substrate material 12′ modified concentration for promoting the sealing accumulation of a biological object O to be analyzed on the aperture 14 and for increasing the residual or sealing resistance is a core aspect of the present invention.

An additional or alternative core aspect of the present invention is that the concentration of the at least one material species or ion species may be modified up to a depth Δ of about 10 nm from the top side 12a or the surface of the carrier 12 and in particular from the aperture inner wall 11i.

The aspects according to the present invention of the lateral locality—e.g. to the aperture region 10 or to a region 20 of the aperture region 10—and of the depth Δ of about 10 nm with respect of the concentration modification may be or are realized separately or together.

The wall region 11 as such forms according to the embodiments of FIGS. 1A and 1B a closed wall extending according to an inner curved surface area having an inner side 10i, 11i or inner wall 10i, 11i and an outer side 10a, 11a or outer wall 10a, 11a. In this manner an aperture 14 of the aperture region 10 of the electrophysiological measuring arrangement 100 according to the present invention is formed, wherein the inner side or inner wall 10, 11i of the wall region 11 faces the aperture 14, and the outer wall or outer side 10a, 11a of the wall region 11 is averted from the aperture 14 but faces the compartment 40 of the electrolyte bath 40, 60, 70.

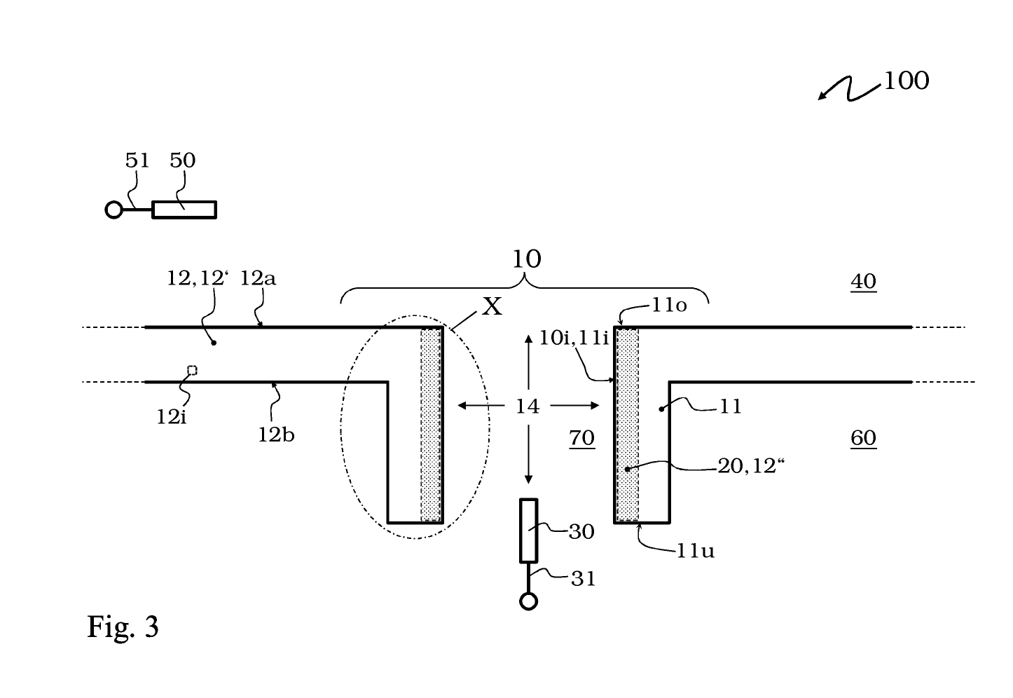

In the embodiment of FIG. 1A the wall region 11 protrudes only above the top side 12a of the carrier or substrate 12. On the bottom side 12b of the substrate or carrier 12 the aperture region 10 is formed quasi planar. Such a structure is however not mandatory and FIGS. 3 to 7 show respectively modified embodiments, which will be described later in detail.

As has been mentioned above the wall region 11, which forms the aperture 14, is formed according to a curved surface area of a geometrical body. According to FIG. 1B together with FIG. 1A this curved surface area may be taken from an upright standing circular cylinder as basic form.

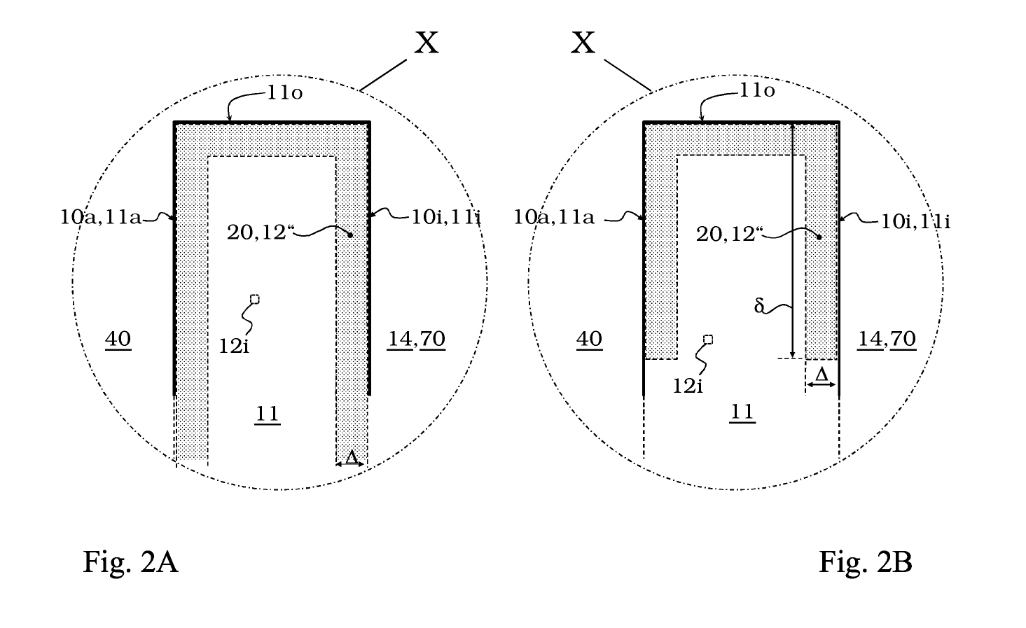

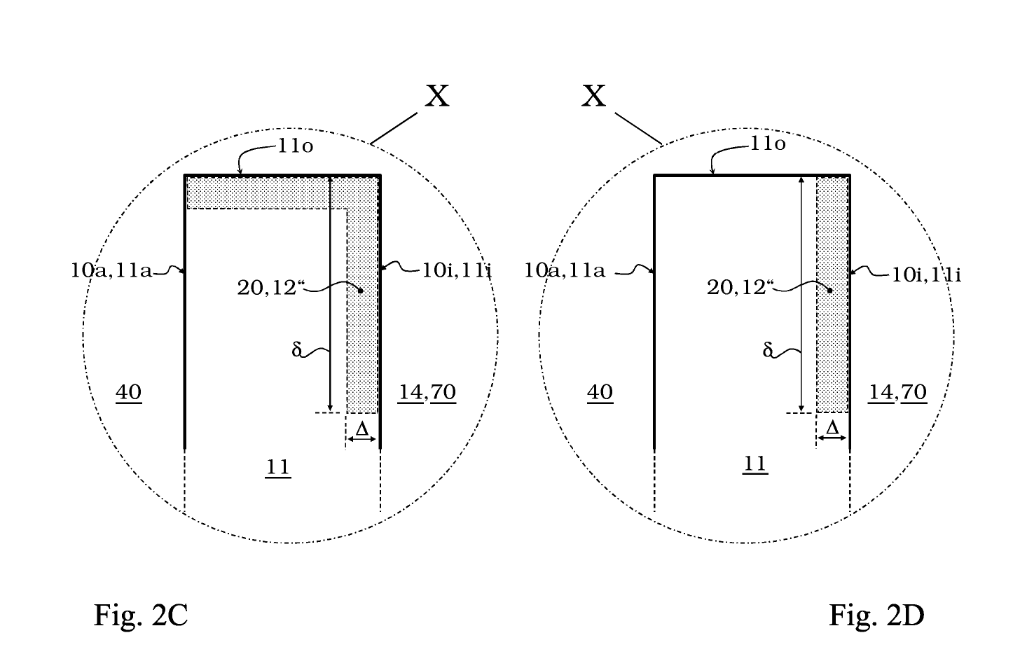

In FIGS. 1A and 2B the wall region 11 or the aperture wall 11 is not formed completely modified with respect to the concentration. The concentration modified region 20 and the aperture wall 11 or the wall region 11 is not fully coincident in this embodiment. In contrast, the concentration modification of the at least one material species or ion species and hence the concentration modified region 20 extend only up to a depth Δ of about 10 nm viewed from the inner wall 11i of the aperture wall 11.

Further alternatives will be described below in connection with FIGS. 2A to 2D.

The form of a curved surface area of an upright standing circular cylinder as basic form is not mandatory. FIGS. 1C and 2D show in schematic top view instead of a cylinder form as base area a square resulting three-dimensionally in an upright quadratic prism, or a prism with a base area in form of an oval, quasi of a square or rectangle with rounded corners, as is illustrated in FIG. 1D. Also in this cases the concentration modifications of the at least one material species or ion species and hence the concentration modified region 20 extend only up to a depth Δ of about 10 nm viewed from the inner wall 11i of the aperture wall 11, too.

Principally, arbitrary forms of base areas are conceivable. However, due to the circumstances described above according to which surface roughness and sharp edges should be avoided, straight forms with rounded structures are preferable, i.e. for example the design according to FIG. 1B which has an upright circular cylinder as geometrical basic form.

In the embodiment of FIG. 1E lateral locality of the concentration modified region 20 and hence restriction to the aperture region 10 and in particular to the aperture wall 11 are set aside. Here, the complete top side 12a of the carrier 12 and the aperture inner wall 11i are concentration modified up to a depth Δ of about 10 nm in their concentration of the at least one material species or ion species.

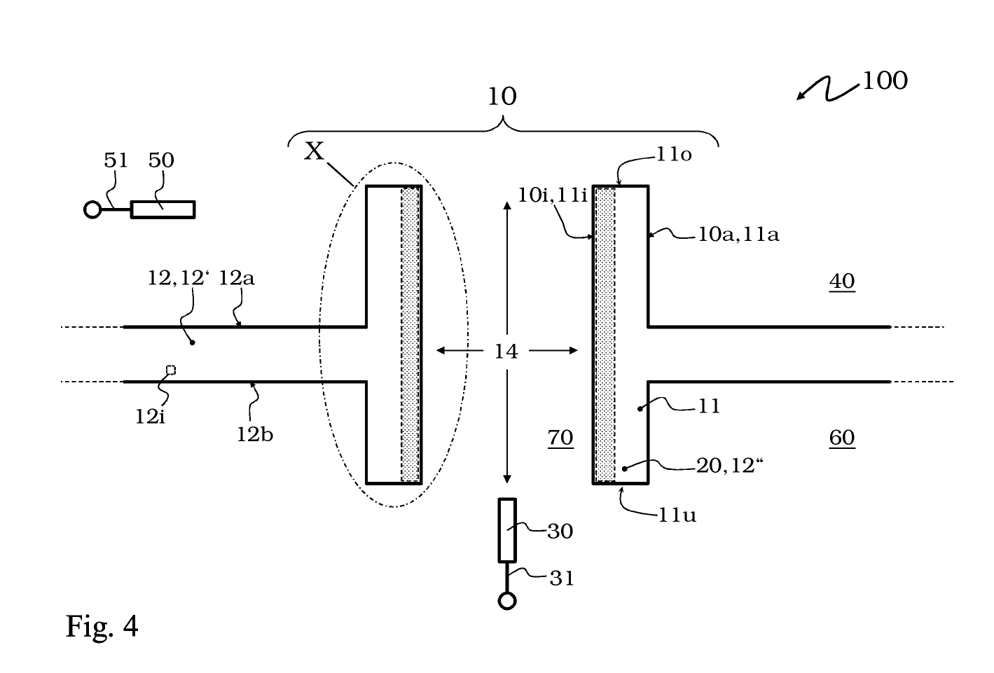

FIGS. 3 and 4 show in analogy to FIG. 1A and also in schematic and sectional side view other embodiments of the measuring arrangement according to the present invention, in which there is a difference in that according to FIG. 3 the wall region 11 for the aperture 14 terminates flush with the top side 12a of the substrate 12 and protrudes only over the bottom side 12b of the substrate 12 such that altogether a kind of an inversion of the top side 12a towards the inside and the compartment 60 is realized for the aperture 14.

In FIG. 4 a part of the wall region 11 of the aperture 14 extends from the top side 12a of the substrate 12 into the compartment 60, however, on the other hand also a part of the wall region 11 extends from the bottom side 12b of the substrate 12 into the compartment 60.

The heights above the top side 12a and above the bottom side 12b by which the wall region 11 for the aperture 14 extends respectively may be identical. However, this is not mandatory. In FIG. 4 they are formed differently.

In the embodiments of FIG. 1A as well as 3 and 4 the cross-section or diameter along the extension direction of the wall region 11 perpendicular to the top side 12a or perpendicular to the bottom side 12b of the substrate is constant in its distribution.

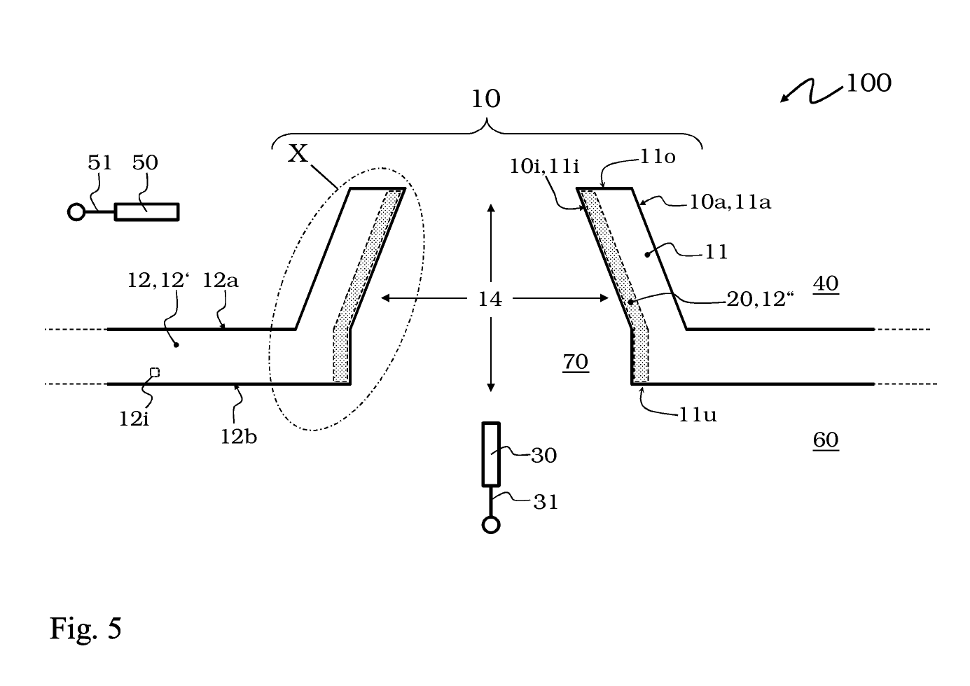

Alternatively tapering or extending cross-sectional distributions may be provided. This is illustrated in the sequence of FIGS. 5 to 7, wherein FIG. 5 shows an embodiment of the measuring arrangement according to the present invention, which corresponds to the embodiment of FIG. 1, however, with a cross-sectional distribution of the aperture 14, which tapers with increasing distance from the top side 12a.

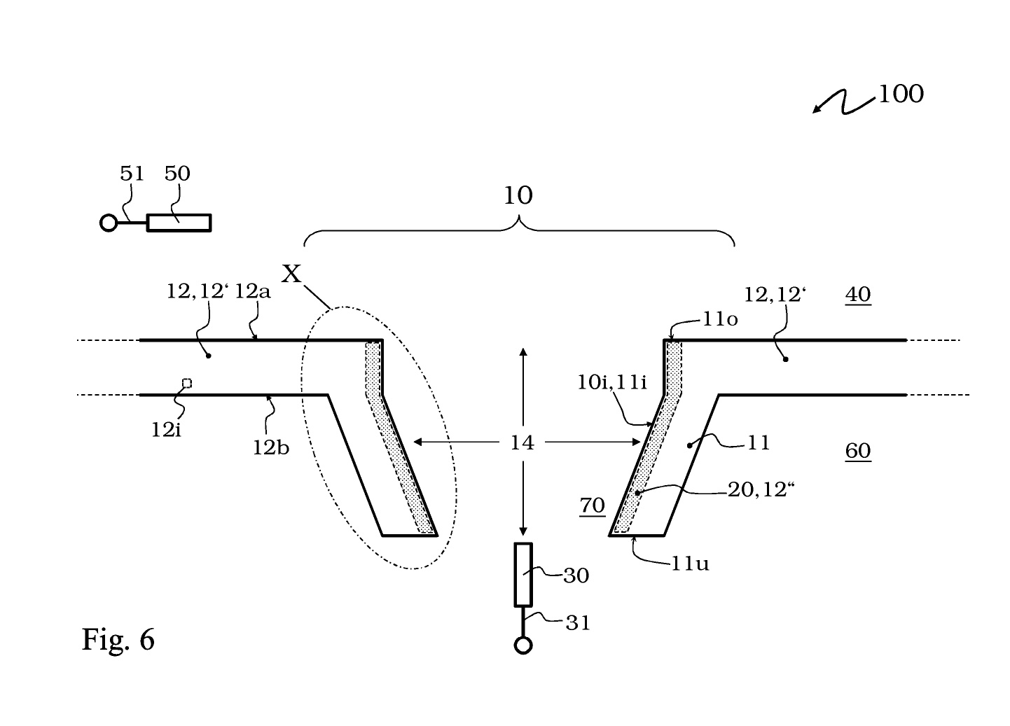

In the embodiment of FIG. 6 the aperture 14 is analogously and in comparison to the embodiment of FIG. 3 formed such that the cross-sectional distribution tapers with increasing distance from the bottom side 12b of the substrate 12.

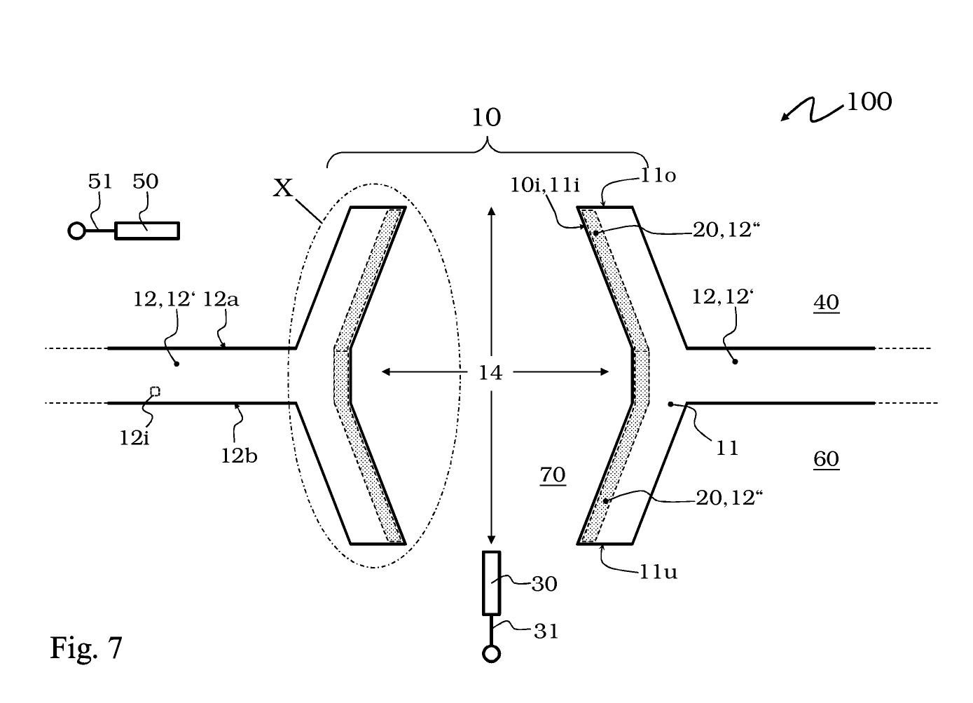

In combination of the embodiments of FIGS. 5a and 6 and in analogous view to the embodiment of FIG. 4 an aperture 14 is shown in FIG. 7, for which the diameter of the aperture 14 is maximal at the height of the substrate 12 and tapers with increasing distance to the top side 12a of the substrate 12 as well as to the bottom side 12b of the substrate 12.

According to the embodiment of FIG. 1A the measuring electrode 30 with the connection or line 31 is formed in close neighborhood and partially inserted into the aperture region 10 with the aperture 14. The position of the measuring electrode 30 may vary, it may for example be inserted even more into the electrolyte region 70 in the aperture 14 or may be farther apart thereof.

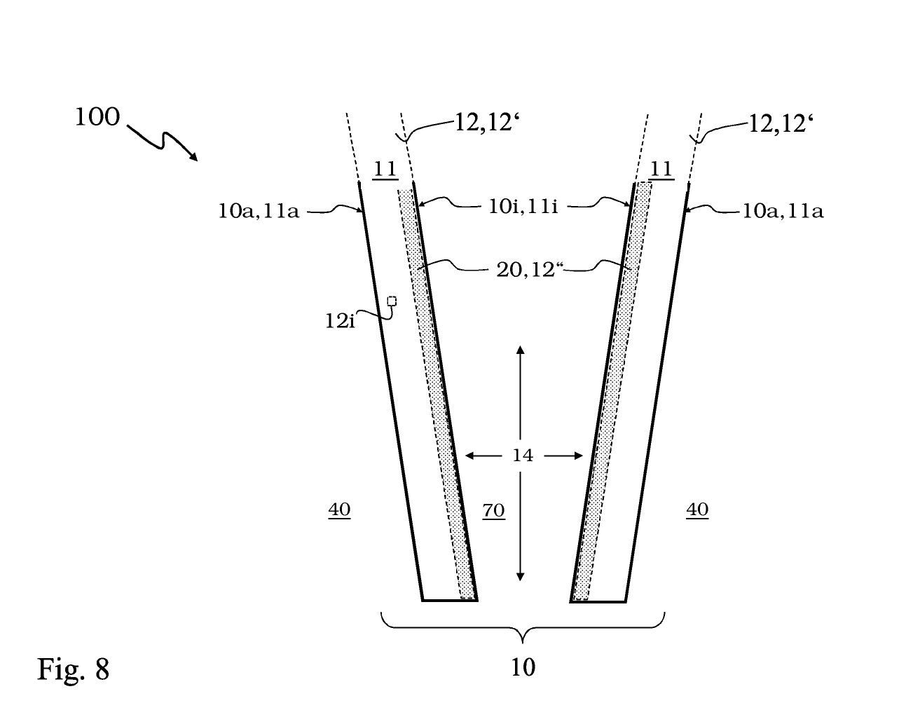

FIG. 8 shows in schematic and sectional side view an embodiment of the electrophysiological measuring arrangement 100 according to the present invention, in which the aperture region 10 is formed by a wall region 11, which is formed in the manner of a patch pipette, altogether.

Also here an inner wall region 10i, 11i, which faces the aperture 14, as well as an outer wall region 10a, 11a are provided, wherein the outer wall region 10a, 11a faces during application an electrolyte compartment 40 of the bath 40, 60, 70 lying outside and wherein the inner wall region 10i, 11i faces the electrolyte compartment 70 of the bath 40, 60, 70 that is provided within the aperture 14. In the outermost region the wall region 11 forming the aperture 14 is formed, which carries the modified region 20 with the material 12″ with the modified or increased concentration of the at least one material species or ion species, and which can hence support the sealing accumulation of a biological object O to be analyzed.

FIGS. 2A to 2D show in more detail the portion X of FIG. 1A and various modifications regarding the design of the modified region 20 with the material 12″ with the modified or increased concentration of the at least one material species or ion species.

In FIG. 2A the concentration modified region 20 with the concentration modified material 12″ extends across the complete upper section of the wall region 11. The change of concentration concerns here a layer up to a depth Δ of about 10 nm in the inner wall 11i of the aperture wall 1 and its outer wall 11a.

In the embodiment according to FIG. 2B the concentration modification with the layer thickness Δ is present only up to a specific distance or height δ from the upper edge 11o or the upper border 11o of the aperture wall 11.

In FIG. 2C only the most upper region of the inner side 10i, 11i and the most upper section up to a layer thickness Δ are concerned and modified in its concentration.

In the embodiment according to FIG. 2D only the inner side 10i, 11i is concerned up to a layer thickness Δ and modified in its concentration.

FIGS. 9A to 9E show in schematic and sectional side view different measuring principles, which may be used in the electrophysiological measuring arrangement 100 according to the present invention and the corresponding electrophysiological measuring method according to the present invention.

Starting from the situation illustrated in FIG. 9A in which by a slight aspiration of the electrolyte bath 40, 60, 70 through the measuring aperture 14, i.e. with a suction from the compartment 40 via the compartment 70 in the aperture 14 to the compartment 60, a biological object O, in this case for example a cell, is aspirated and brought closer to the aperture 14.

The approximation mechanism may also be carried out in another manipulative manner for example by means of a separate pipette, a laser forceps or the like.

By means of a slight under-pressure the cell is accumulated completely and without destruction on the measuring aperture 14 according to FIG. 9B. A measurement in this state is denoted as so-called cell-attached-mode.

By a mechanic tension starting from the situation described with respect to FIG. 9B a part of the membrane may be ripped out of the membrane of the biological object O such that according to FIG. 9C the ripped out part of the membrane, a so-called patch, remains in a sealed state in the measuring aperture 14 and constitutes the actual measuring object O. Here, due to the ripping out the side of the part of the membrane arranged formally in the inside is now directed to the outside, i.e. towards the compartment 40. Therefore this measuring mode is also called inside-out-mode.

On the other hand, starting from the situation illustrated in FIG. 9B, by applying again under-pressure, namely e.g. in the manner of a pressure pulse the cell O may be opened as a whole such that a transition from the so-called cell-attached-mode to the whole-cell-mode according to FIG. 9D occurs, in which the measuring electrode 30 has direct access to the whole inside of the cell. This means the inside of the cell is opened towards the compartment 70 and 60, but is substantially isolated from the compartment 40.

Starting from the situation according to FIG. 9D, i.e. from the whole-cell-mode, it may be achieved by mechanic tension that again a membrane fragment is ripped out from the membrane of the biological object. As previously the whole-cell-mode was present there is a certain probability that the ripped out part of the membrane remains such in the measuring aperture 14 that is can serve as actual measuring object O and that according to FIG. 9E the outer side of the cell membrane or the membrane of the biological object remains still outside. This case is called an outside-out-mode of the measuring arrangement 100.

FIG. 10 shows in detail the geometric situation, which occurs for a sealing accumulation of a biological object O to be measured between its membrane M and the measuring aperture 14 and in particular the inner wall 10i, 11i of the wall region 11. The foremost section of the wall region 11 is formed by the concentration modified region 20 with or from the material 12″ with the modified concentration.

FIG. 10 shows again a whole-cell-mode, in which the whole cell O is opened with its inside towards the measuring electrode 30 and the electrolyte compartment 60 and 70. This embodiment of the electrophysiological measuring arrangement 100 according to the present invention is here formed in the manner of a patch pipette.

According to the present invention the sealing resistance between the cell membrane M of the biological object O to be measured and the inner wall 10i, 11i of the wall region 11 of the measuring aperture 14 is improved by providing the concentration modified region 20 with or from the material 12″ with modified concentration and by interaction between the concentration modified region 20 with the outer side cell membrane M of the biological object O.

According to the present invention it is hence possible, by selection of the modification of the concentration in the concentration modified region 20 according to type and strength, to take into account different situations on different surfaces of membranes M, which may be cell membranes, membranes of organelles, or membranes of artificial objects. This was up to now not possible in this manner and provides a possibility to promote accumulation and a seal in a controlled manner or to stabilize a seal, if it is formed.

In the embodiment according to FIG. 11 the substrate is quasi covered by a layer of cells O′. By mechanical and/or electrical interaction under use of the concentration modified region 20 provided according to the present invention inside the aperture wall 11 with or from the material 12″ with modified concentration, a single cell O of the ensemble of cells O′ is available as measuring object in the whole-cell-mode in analogy to the situation according to FIG. 9D with an improved sealing accumulation for an electrophysiological measurement.

FIG. 12 shows an arrangement of the measuring arrangement 100 according to the present invention, in which the wall region 11 forming an aperture 14 is formed by an edge or edge region such that the aperture 14 is quasi formed as a planar hole in the underlying substrate 12, wherein the concentration modified region 20 with or from the material 12″ forms the edge region with an accordingly modified concentration of at least one material species or ion species to promote accumulation and to increase the sealing resistance.

### LIST OF REFERENCE SIGNS

10 aperture region
10a outer wall, outer wall region, outer side of the aperture region 10
10i inner wall, inner wall region, inner side of the aperture region 10
10i wall region, wall, aperture wall
11a outer wall, outer wall region, outer side of the wall region 11
11i inner wall, aperture inner wall, inner wall region, inner side of the aperture region 11
11o upper rim/upper edge outer wall, outer wall region, outer side of the aperture wall 11
11u bottom rim/bottom edge outer wall, outer wall region, outer side of the aperture wall 11
12 substrate, carrier, base, base substrate
12′ carrier material or substrate material (with normal concentration)
12″ material, carrier material or substrate material with modified, in particular increased concentration of a material species or ion species
12a top side, front side
12b bottom side, back side
12i inside of the carrier 12, inner region of the carrier 12, bulk of the carrier 12
14 aperture, measuring aperture
20 region with modified, in particular increased concentration; concentration modified region
30 measuring electrode, measuring electrode arrangement
31 measuring line, line
40 electrolyte compartment, electrolyte bath
50 counter electrode, counter electrode arrangement
41 line
40 electrolyte compartment, electrolyte bath towards the top side 12a of the carrier 12
60 electrolyte compartment, electrolyte bath towards the back side 12b of the carrier 12
70 electrolyte compartment, electrolyte bath inside the lumen of the aperture 14
100 electrophysiological measuring arrangement
M membrane of the biological object O
O biological object, cell, liposome, vesicle, micelle, oocyte, measuring object
O′ biological object, cell, liposome, vesicle, micelle, oocyte
δ height of the concentration modified region 20 in the aperture wall 11
Δ depth or layer thickness of the concentration modified region 20

1-10. (canceled)

11. An electrophysiological measuring arrangement, comprising:
a carrier comprising a carrier material with a specific composition at least in an inside of the carrier; and
an aperture region in a region of the carrier configured for controlled sealing accumulation of a biological object,
wherein the aperture region comprises an aperture and a wall region which surrounds the aperture, the wall region having an aperture inner wall which defines the aperture,
wherein at least the wall region is formed with a region made of a material which corresponds substantially to the carrier material at least in its composition, but has in comparison to the carrier material at least one material species or ion species with an increased concentration,
wherein the concentration of the at least one material species or ion species is modified up to a predetermined depth from a top side and/or surface of the carrier and from the aperture inner wall up to a depth of about 10 nm,
wherein the at least one material species or ion species comprises double charged metal cations.

12. The electrophysiological measuring arrangement of claim 10, wherein the increase of concentration of the at least one material species or ion species is spatially locally limited to a region of the wall region, such that the carrier material does not have an increased concentration of the at least one material species or ion species at least in one region outside of the aperture region.

13. The electrophysiological measuring arrangement of claim 10, wherein the concentration of the at least one material species or ion species is formed to be increased by means of implantation.

14. The electrophysiological measuring arrangement of claim 12, wherein the concentration of the at least one material species or ion species is formed to be increased by means of a plasma process, a sputter process or an ion beam process.

15. The electrophysiological measuring arrangement of claim 10, wherein the aperture region is formed in a region of the carrier between the top side and a bottom side of the carrier, and wherein the wall region which surrounds the aperture protrudes with respect to the top side and/or with respect to the bottom side of the carrier.

16. The electrophysiological measuring arrangement of claim 10, wherein the wall region which surrounds the aperture has a curved surface area or a combination of curved surface areas.

17. The electrophysiological measuring arrangement of claim 15, wherein the wall region which surrounds the aperture has a curved surface of at least one of a cylinder, a prism, a truncated cone and a truncated pyramid.

18. The electrophysiological measuring arrangement of claim 10, wherein the wall region which surrounds the aperture comprises a material selected from the group consisting of: glass; quartz glass; silicon; carbon and combinations and derivatives thereof.

19. The electrophysiological measuring arrangement of claim 10, wherein a diameter of the aperture is between 1 μm and 50 μm.

20. The electrophysiological measuring arrangement of claim 10, wherein the wall region which surrounds the aperture has a height above the top side or below a bottom side of the carrier of 20 μm or less.

21. The electrophysiological measuring arrangement of claim 10, further comprising:
a measuring electrode provided within the aperture or in a region on a bottom side of the carrier; and
a counter electrode provided outside of the aperture and in a region above the top side of the carrier.

22. An electrophysiological measuring method using an electrophysiological measuring arrangement which includes a carrier comprising a carrier material with a specific composition at least in an inside of the carrier and an aperture region in a region of the carrier configured for controlled sealing accumulation of a biological object, the aperture region comprising an aperture and a wall region which surrounds the aperture, the wall region having an aperture inner wall which defines the aperture, at least the wall region being formed with a region made of a material which corresponds substantially to the carrier material at least in its composition, but has in comparison therewith at least one material species or ion species with an increased concentration, the concentration of the at least one material species or ion species being modified up to a predetermined depth from a top side and/or surface of the carrier and from the aperture inner wall up to a depth of about 10 nm, the at least one material species or ion species comprising double charged metal cations, the electrophysiological measuring method comprising:
introducing a biological object to be measured; and
controlling the sealing accumulation of the biological object on the aperture of the aperture region.
